(19) United States
(12) Patent Application Publication
(10) Pub. No.: US 2005/0055746 A1
(43) Pub. Date: Mar. 10, 2005

(54) Method for increasing protein content in plant cells

(75) Inventors: Dominique Michaud, Quebec (CA); Raphael Anguenot, Quebec (CA); France Brunelle, Quebec (CA)

Correspondence Address: OGILVY RENAULT, 1981 MCGILL COLLEGE AVENUE, SUITE 1600, MONTREAL, QC, H3A2Y3, CA

(73) Assignee: Universite Laval, Quebec (CA)

(21) Appl. No.: 10/739,923

(22) Filed: Dec. 19, 2003

Related U.S. Application Data
(60) Provisional application No. 60/434,826, filed on Dec. 20, 2002.

(51) Int. Cl. A01H 1/00; C12N 15/82

(52) U.S. Cl. 800/288

(57) ABSTRACT

The invention relates to a method for the production of plants with increased protein content in leaves, by means of introduction of recombinant DNA molecules. Said recombinant DNA molecules are introduced into the plant, by means of a transformation system and comprise a DNA sequence of plant origin, expressed in plants, the genetic product of which inhibits a protein in leaves with the enzymatic activity of an aspartate protease and/or a serene protease. The plants which displays an increased content in leaf protein are chosen.

### CROSS-REFERENCE TO RELATED APPLICATIONS

This application claims priority on U.S. provisional application No. 60/434,826.

### TECHNICAL FIELD

The present invention relates to a new method for increasing the net amount of proteins in cells. A particular aspect of the present invention relates to a method allowing to increase the amount of whole proteins in plant cells and tissues.

### BACKGROUND OF THE INVENTION

Proteins are important nutrients necessary for the building, maintenance and repair of animal tissues. Nine of the twenty amino acids constituting proteins cannot be produced by most animals and must be obtained from the diet (Rawn, 1989). A variety of grains, seeds, legumes and vegetables can provide all the essential amino acids needed. Plant leaf proteins can supply large quantities of protein for animal feeds, and can offer a useful potential source of protein for human consumption.

A large proportion of agricultural surfaces are used for animal feeding, in the form of forages or cereals, peas and rapeseed. Pastures and forages are not always sufficient for productive animals that need more proteins, so that food complements are provided by leaf protein concentrates and by-products of the manufacture of vegetable oils. In this context, leaf protein level is an important criterium of crop nutritional quality. Plant breeders aim to select new varieties with higher protein content and appropriate amino acid compositions. However, protein content in a given plant species or tissue is under the control of multiple genetic traits related to various cellular and physiological processes including development, photosynthesis, carbon translocation and respiration.

Proteins are continuously synthesized and degraded in all living organisms, with half-lives ranging from as short as a few minutes to, weeks or more. The concentration of any individual protein is determined by the balance between its rates of synthesis and degradation, which in turn are controlled by a series of tightly-regulated biochemical mechanisms. Proteolysis in plant cells serves a variety of roles, including the control of cell cycle, the recycling of amino acids (e.g. seed germination), the degradation of polypeptides not folded properly, the elimination of foreign proteins, and many other cellular processes (Callis 1995; Estelle 2001). While proteolytic enzymes play vital roles ill vivo, however proteolysis by plant proteases is a severe problem affecting the nutritional quality of crops, notably during the ensiling process (McDonald 1981), or daring the preparation of leaf protein concentrates (Jones et al. 1995).

Proteolysis also is a problem in “molecular farming” systems devised to produce clinically-useful proteins in plants. Plant “biofactories” offer several advantages for the production of heterologous proteins, including notably low production costs, capacity to easily increase production areas based on current plant production systems, possibility of expressing proteins with complex post-translational modifications, and minimal risks for human pathogen contamination (Doran 2000; Daniell and Streatfield 2001; Hood et al. 2002; Ma et al. 2003). It is known from the art, however that plant proteases may alter drastically the stability of foreign (heterologous) proteins in planta (Michaud et al. 1998). Even when all other transcriptional and post-transcriptional processes are optimized for recombinant gene expression (Kusnadi et al. 1997), proteolysis still represents an important barrier affecting protein yields in plant systems (Michaud and Yelle 2000).

At present, the most considered strategy to avoid unwanted proteolysis in planta consists in directing the accumulation of recombinant polypeptides in alternative cellular locations using appropriate targeting signals (Michaud et al. 1998). While several reports suggest that most “non-specific” proteases in plants are cysteine and aspartate proteinases found in the vacuolar compartment (Callis 1995), it is also well established that the ubiquitin pathway implicated in the breakdown of short-life proteins takes place primarily in the cytoplasm and the nucleus (Vierstra 1996). Adding peptide signals to the primary sequence of recombinant proteins to direct their accumulation in extracellular compartments or in the endoplasmic reticulum (ER) is an alternative to prevent degradation (e.g., Wandelt et al. 1992). To this end, the fusion of various peptidic signals to recombinant proteins using appropriate gene constructs has proven functional to specifically control their final destination in transgenic plant cells (Michaud et al. 1998).

Protein stability can also be engineered by removing short amino acid domains involved in the control of protein turnover. This strategy has been used successfully with cyclin, where removal of the N-terminal domain KFERQ resulted in a permanent stabilization of the protein (Glotzner et al. 1991). This strategy, however required a modification of the recombinant protein sequence, and can hardly be used for the stabilization of endogenous proteins.

Another genetic engineering approach to produce plants with increased protein content in seeds is to introduce into these plants a DNA sequence encoding the small subunit of a ADP-glucose pyrophosphorylase (AGP). The expression of the AGP-encoding cDNA sequence in the antisens orientation, under the control of the seed-specific legumin B4 promoter, was shown to result in increased content of total nitrogen and protein (Weber et al. 2000). It was proposed that a higher water uptake consecutive to an accumulation of soluble compounds in transgenic cotyledons led to a better uptake of amino acids, which became available for protein biosynthesis.

In plants, little is known about the interactions between recombinant proteins and intracellular proteases, but the occurrence of hydrolytic processes similar to those observed in bacterial and yeast cells appears likely. Some peptidases found in E. coli or yeast, for instance may rapidly cleave recombinant proteins, stressing the importance of developing efficient strategies to minimize unwanted hydrolytic processes in these organisms. Negative mutant strains of E. coli deficient in various cell envelope proteases have been developed to overcome proteolytic degradation of secreted recombinant proteins (Chistyakova and Antonov 1990; Meerman and Georgiou 1994). In contrast with bacteria and yeast (Gottesman 1990; Cregg et al. 1993), the resident proteases of plant cells have not been thoroughly characterized, and mutants lacking proteases potentially damaging to recombinant proteins are not yet available.

The expression of recombinant PIs in plants has been proposed as a way to protect plants from their natural enemies, and several plants of economic importance were genetically modified with PI-encoding cDNA sequences over the last fifteen years (see Michaud 2000, and Chapters therein). At this point, most studies on PI-expressing transgenic plants documented the potential of recombinant PIs to inhibit extracellular proteases of target pests, but no study considered the possibility of using such inhibitors to control endogenous proteolysis in vivo for enhancing net protein content in cells and tissues during the plant growth period.

Previous studies reported some apparent indirect (pleiotropic) effects of a cysteine PI from rice expressed in transgenic Solanaceae species (Guttericz-Campos et al. 1999; Simon et al. 2000; van der Vyver et al. 2003), suggesting possible metabolic interference of this inhibitor on the host plane's metabolism. From the state of the art, it clearly appears that a method allowing to enhance protein content in plant cells would be highly desirable.

### SUMMARY OF THE INVENTION

One object of the present invention is to provide a method for increasing total protein content in a plant cell. In one embodiment, the method comprises inducing the plant cell to recombinantly produce a modifier of protein metabolism.

In one embodiment of the method of the present invention the modifier alters the overall rate of cellular proteolysis. The modifier may be a protease inhibitor. This decrease preferably occurs in the cytoplasm of the plant cells.

According to the present invention, protein content can be increased by about 10 to 50% when compared to the protein content of a plant cell in which the modifier is not recombinantly produced.

To produce the modifier in a recombinant manner, the plant cell is a genetically modified plant cell. The genetic modification can be either genomic or episomic.

Another aim of the present invention is to provide a plant cell or a plant which recombinantly produces a modifier of protein metabolism to increase the content of protein within the plant cell or the plant.

This invention may advantageously be used to improve the agronomic value of any plant used as forage, and/or used as a source of protein for animal feeds and/or human consumption, without notable interference with growth and development of the plant. In addition, the invention may represent a powerful approach to circumvent the loss of leaf proteins under limiting growth conditions, like low light intensities. This approach may also be used to limit the degradation of heterologous and endogenous proteins by proteases in planta, and thus improve yields of heterologous and/or total proteins recovered from the plant. Other uses, as would be obvious to one skilled in the art, are also contemplated as being part of the invention.

### BRIEF DESCRIPTION OF THE DRAWINGS

Further features and advantages of the present invention will become apparent from the following detailed description, taken in combination with the appended drawings, in which:

FIG. 1 shows variable expression of the tomato cdi transgene in selected control (non-transgenic [K] and transgenic [SPCD]) and CDI-expressing (transgenic; “CD” lines) plant lines, as determined by northern blotting using a DNA probe for cdi.

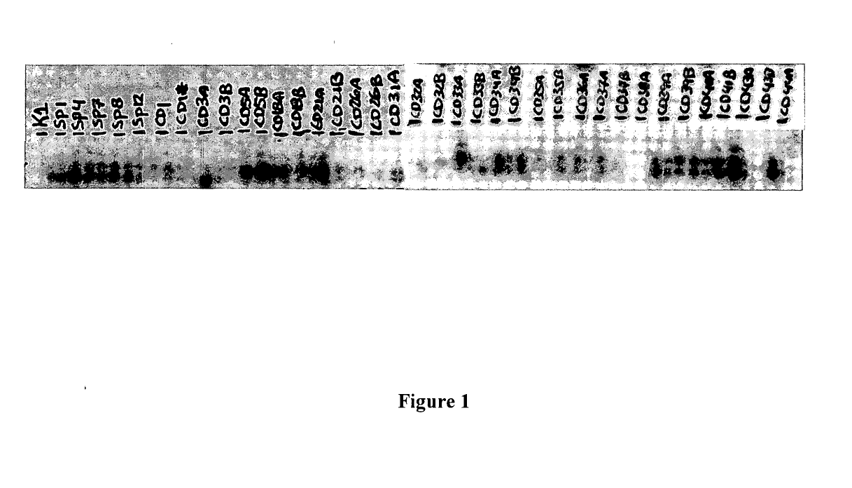

FIG. 2 shows expression of the tomato cdi transgene and accumulation of recombinant CDI in control (K, SpCD7) and CDI-expressing transgenic clones. A: northern blot showing cdi mRNA levels in some of the clones. B: Detection of active recombinant CDI on dot blot, as detected by functional immunodetection with human cathepsin D as a target enzyme. Bars on panel B show the relative amount of recombinant CDI on blot, compared to the amount observed for clone CD21A, fixed arbitrarily at 1.0. Each bar is the mean of three values±se.

FIG. 3 illustrates the growth curve of control (K, SpCD7) and CDI-expressing potato clones emerged from tubers after sowing in a growth chamber. Data are expressed as plant heigth (cm) over time. Each point is the mean of 6 values±se.

FIG. 4 illustrates total leaf protein content in control and transgenic potato plants grown under elevated light intensities. Protein content was assayed for the 4, leaf of transgenic potato lines expressing low (+) or high (+++) levels of recombinant tomato CDI. Each value is the mean (±SE) of three replicates.

FIG. 5 illustrates total leaf protein content in control and transgenic potato plants grown under low light intensities. Protein content was assayed for the 4^{th} leaf of transgenic potato lines expressing low (+) or high (+++) levels of recombinant tomato CDI. Each value is the mean (±SE) of three replicates.

### DETAILED DESCRIPTION OF THE PREFERRED EMBODIMENT

For the purpose of the present invention the following terms are defined below.

The expression “genetically modified”, as used herein, is intended to mean transgenic cells or plant which exhibit the transgene in all cell, or that are chimeric i.e exhibiting a pattern of transgenic cells and non-transgenic cells. Thus, chimeric plants would exhibit a transgene in some cells, groups of cells, or parts of the whole plant.

The expressions “recombinantly produced” or “recombinant protein”, as used herein, are intended to mean production of a protein, or peptide encoded by a DNA sequence that is either homologous or heterologous (transgene) to the native genome of the cell or plant cell in which it is produced in accordance with the present invention.

Heterologous gene or DNA is a A DNA sequence that encodes a specific product fulfilling a biological function, and that originates from a species other than that into which the said gene is to be inserted; the said DNA sequence is also referred to as a foreign gene or trans gene.

Homologous gene or DNA refers to a DNA sequence that encodes a specific product fulfilling a biological function, and that originates from the same species as that into which the said gene is to be inserted.

By the term modifier of protein metabolism it is meant a protein (or peptide) capable of modifying the overall protein synthesis/degradation (proteolysis) balance, thereby influencing the protein content of a cell. It will be appreciated that such a modifier may act at different stages and/or physical locations of protein synthesis or degradation pathways either directly by affecting a component that exerts a direct control of protein synthesis of degradation, or indirectly by affecting a component affecting a component that in turn affects a component that exert a direct control.

Plant promoter refers to a control sequence for DNA expression that ensures the transcription of any desired homologous or heterologous DNA gene sequence in a plant, in so far as the said gene sequence is linked in operable manner to such a promoter.

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

The invention presented herein constitutes a new method to substantially increase protein content in host organisms such as multicellular eukaryotes. More particularly, the present invention is designed to be applied in plant cells, such as, but not limited to, cells of leaves. This can be achieved by transforming the plant cell with a genetic construct that comprises a promoter and a gene coding for a modifier of protein metabolism, such that the expression of this modifier increases the protein content of a cell.

The gene encoding the modifier is preferably inserted into a genetic construct that comprises a promoter compatible with gene expression in the intended recipient organism. Those skilled in the art will recognize that there are a number of promoters which are active in plant cells, and have been described in the literature. Such promoters may be obtained from plants, plant viruses, or plant commensal, saprophytic, symbiotic, or pathogenic microbes and include, but are not limited to, the nopaline synthase (NOS) and octopine synthase (OCS) promoters (which are carried on tumor-inducing plasmids of Agrobacterium tumefaciens), the cauliflower mosaic virus (CaMV) 19S and 35S promoters, the light-inducible promoter from the small subunit of ribulose 1,5-bisphosphate carboxylase (ssRUBISCO), the rice Act1 promoter, the Figwort Mosaic Virus (FMV) 35S promoter, the sugar cane bacilliform DNA virus promoter, the ubiquitin promoter, the peanut chlorotic streak virus promoter, the comalina yellow virus promoter, the chlorophyll a/b binding protein promoter, and the meristem enhanced promoters Act2, Act8, Act11 and EF1a and the like. All of these promoters have been used to create various types of DNA constructs which have been expressed in plants (see e.g., McElroy et al., 1990, U.S. Pat. No. 5,463,175; Barry and Kishore, U.S. Pat. No. 5,463,175) and which are within the scope of the present invention. Chloroplast and plastid specific promoters, chloroplast or plastid functional promoters, and chloroplast or plastid operable promoters are also envisioned. It is preferred that the particular promoter selected should be capable of inducing sufficient in planta expression to result in the production of an effective amount of the modifier of the present invention, such as to increase to protein content of a cell.

One set of preferred promoters are constitutive promoters such as the CaMV35S or FMV35S promoters, that yield high levels of expression in most plant organs. In addition, it may also be preferred to bring about expression of the modifier in specific tissues of the plant, such as leaf, stem, root, tuber, seed, fruit, etc., and the promoter chosen should have the desired tissue and developmental specificity. Therefore, promoter function should be optimized by selecting a promoter with the desired tissue expression capabilities.

In a preferred embodiment of the invention the modifier alters the overall protein synthesis/degradation balance and leads to increased protein content in cells. In this case, the modifier is preferably a protease inhibitor such as, but not limited to, an aspartate protease inhibitor (e.g., a oathepsin D inhibitor), a cysteine protease inhibitor (e.g., a cystatin), a serine protease inhibitor (e.g., a trypsin or chymotrypsin inhibitor), or a metalloprotease inhibitor.

The modifier is preferably expressed and secreted in the cytoplasm, thereby affecting protein metabolism in the cytoplasm. However, it may be particularly advantageous to direct the localization of the modifier to one or more subcellular compartments, for example to the mitochondrion, the endoplasmic reticulum, the vacuole, the chloroplast or any other plastidic compartment. For example, proteins can be directed to the chloroplast by including at their amino-terminus a chloroplast transit peptide (CTP). Accordingly the protein content of such cellular compartments may be selectively increased. Signal sequences responsible for compartment targeting are well known in the art.

There are many methods for introducing transforming DNA segments into cells, but not all are suitable for delivering DNA to plant cells. Suitable methods are believed to include virtually any method by which DNA can be introduced into a cell such as by Agrobacterium infection, binary bacterial artificial chromosome (BIBAC) vectors, direct delivery of DNA such as, for example PEG-mediated transformation of protoplasts, desiccation/inhibition-mediated DNA uptake, electroporation, agitation with silicon carbide fibers, acceleration of DNA coated particles, etc. In certain embodiments, acceleration methods are preferred and include, for example, microprojectile bombardment and the like.

Transformation of the cell with the genetic construct of the present invention may be effected so that the gene encoding the modifier be inserted in the cell either episomically or genomically.

In another aspect, there is also provided cells and plants genetically modified to incorporate a modifier as described above, and wherein the transgenic cell or plant is capable of producing an increased amount of protein compared to non-transformed cells or plants.

In a preferred embodiment, the host organism is a plant host selected from the group consisting of plant protoplasts, cells, calli, tissues, organs, zygotes, embryos, pollen and/or seeds and also, especially, whole, preferably fertile, plants that have been transformed with the recombinant modifier. Whole plants can either be transformed directly as such with the recombinant DNA molecule according to the invention, or they can be obtained from previously transformed protoplasts, cells and/or tissues by regeneration.

The present invention will be more readily understood by referring to the following examples, which are given to illustrate the invention rather than to limit its scope.

### EXAMPLE 1

Transgenic potato plants expressing tomato CDI, a cathepsin D inhibitor from tomato, Lycopersicon esculentum Mill.

To assess the onset of metabolic interference after ectopic expression of an aspartate protease inhibitor, tomato cathepsin D inhibitor (CD1) on host plant metabolism and endogenous protein stability in planta, two different gene constructs were introduced into potato (Solanum tuberosum cv Kennebec): one expressing the tomato cdi transgene under the control of the cauliflower mosaic virus ³⁵S (CaMV35S) promoter; and one with the same transgene but with no promoter (transgenic controls; “SPCD” lines) (see Brunelle et al. 2004). For SPCD lines, a tomato CDI-encoding DNA sequence was isolated from the expression vector pGEX-3×/CDI (Brunelle et al. 1999) by digestion with BamHI and EcoRI, and subcloned between the BamHI and EcoRI cloning sites of the commercial vector pCambia 2300 (CAMBIA, Canberra, Australia). For CD plants, the CaMV35S promoter was isolated from the commercial plasmid pBI-121 (Clontech, Palo Alto, Calif.) by a BamHI/SalI treatment, and then ligated between the BamHI and SalI cloning sites of the pCambia construct including the cdi transgene (see above).

Axenically-grown plantlets of potato (Solanum tuberosum L. cv. Kennebec) were used as source material for genetic transformation. The plantlets were maintained on MS multiplication medium supplemented with 0.8% (w/v) agar (Difco, Detroit, MT) and 3% (w/v) sucrose, in a tissue culture room at 22° C. under a light intensity of 60 μmol.m⁻².s⁻¹ and a L:D 16:8 h photoperiod provided by cool fluorescent lights. Leaf discs about 10 mm in diameter were genetically-transformed using the bacterial vector Agrobacterium tumefaciens LBA4404 as described by Wenzler et al. (1989), except that cefotaxime, instead of carbenicillin, was used for A. tumefaciens growth control. Regenerated shoots were transferred onto selection medium with kanamycin and cefotaxime, for root regeneration and plantlet multiplication. For acclimation, the plantlets were transferred for 14 days in a growth chamber under a 24′/21° C. day/night temperature cycle, a L:D 12:12 h photoperiod, a light intensity of 200 μmol.m⁻².s⁻¹ and a relative humidity of 60%, before being transferred in greenhouse under standard growth conditions.

About 45 plantlets regenerated from distinct callus sprouts and selected on kanamycin⁺ growth medium were acclimated in the greenhouse and tested for the presence of the nptii and cdi transgenes. Integration of the nptii (marker) transgene in kanamycin-resistant plants was confirmed by PCR, using DNA extracted from the fourth, fifth and sixth leaves [from the apex] of ˜30-cm potato plants, according to Edwards et al. (1991). The following primers were used for amplification: 5′-ACT GAA GCG GGA AGG GAC TGG CTG CTA TTG; and 3′-GAT ACC GTA AAG CAC GAG GAA GCG GTC AG. The transgene was visualized by ethidium bromide staining, after resolving the PCR products (˜500 bases) into 1% (w/v) agarose gels. A 500-bases-long nptii amplicon was amplified by PCR from genomic DNA of all plants tested, confirming that plants regenerated on kanamycin had been genetically transformed by the bacterial vector (not shown).

RT-PCR analysis with primers for cdi (not shown) and northern blotting with a cdi probe were then carried out with total RNA extracted from the fourth, fifth and sixth leaves of nptii transgene-positive plants (Logemann et al., 1987), to confirm expression of the cdi transgene in leaves of plants transformed with the camv35S/cdi construct (“CD” lines). The following primers were used for RT-PCR: 5′-AAG GAT CCG TGC ACA AAA GAT GGC TGC TTC TCC TAA ACC TAA TCC AGT AC; and 3′-AAC CCG GGA AGC CGA GAC TTT CTT GAA GTA GAC CCC CAA G. After amplification, cdi amplicons (600 bases) were visualized by 1% (w/v) agarose gel electrophoresis and staining with ethydium bromide. For northern blotting, RNA was resolved into 1.2% (w/v) formaldehyde-agarose gels and blotted onto nitrocellulose sheets (Hybond™-N⁺, Amersham, Piscataway, N.J.). The blots were hybridized for 20 h at 68° C. with a ³²P-labelled DNA probe corresponding to a PCR amplicon of the cdi transgene, and washed under stringent conditions. The filters were subjected to autoradiography overnight at −80° C., using Kodak Biomax films (Kodak, Rochester, N.Y.) and intensifying screens.

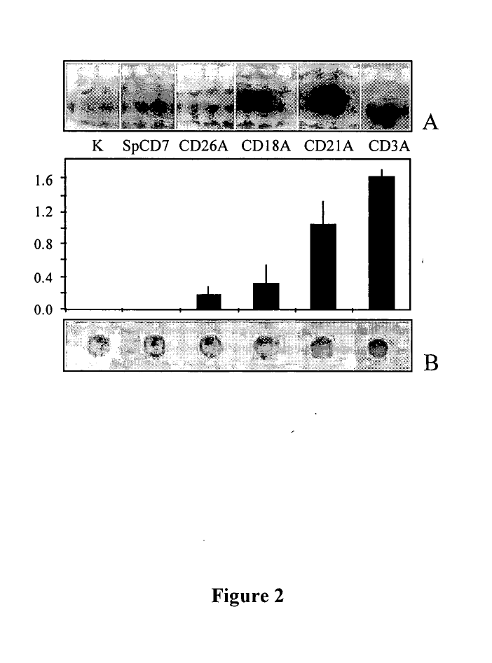

While a faint signal—presumably corresponding to potato endogenous cdi, ˜80% homologous to the tomato CDI coding sequence (Werner et al. 1993)—was detected on blots for non-transgenic (e.g., FIG. 2A, clone K) and transgenic (e.g. clone SpCD7) controls, stronger signals were observed for tomato CDI-expressing clones, with expression levels ranging from low (e.g., clone 26A) to medium (e.g., clone 18A), high (e.g., clone 3A) or very high (e.g., clone 21A).

The amount of active tomato CDI in transgenic potato lines was visualized by functional immunodetection, using human cathepsin D as target enzyme and a mouse monoclonal antibody directed against this enzyme (FIG. 2B). In brief, 25 μg of leaf protein was fixed onto a nitrocellulose sheet using the Bio-Dot Microfiltration Apparatus™ (Biorad), and incubated for 60 min at room temperature with 0.5 μg of human cathepsin D dissolved in 75 μl of 20 mM citrate phosphate, pH 6.0, containing 500 mM NaCl. After incubating membranes with the mouse primary antibody, CDI/cathepsin D complexes were visualized with an alkaline phosphatase-conjugated secondary antibody and appropriate reagents for detection of phosphatase activity. To avoid breakdown of the CDI/cathepsin D complex on nitrocellulose membranes, color development was carried out at pH 7.0, in 100 mM Tris-HCl containing 10 mM NaCl and 5 mM MgCl₂. Leaf proteins were extracted from the fourth, fifth and sixth leaves of potato plants as described earlier (Cloutier et al. 2000), and protein contents were determined according to Bradford (1976) using bovine serum albumin as a standard. As shown on FIGS. 2A and B, differential levels in cdi mRNA resulted in varying amounts of recombinant CDI in leaves of the transgenic clones, with detectable levels of active inhibitor ranging from very low in control lines (clones K1 and SpCD7) to medium and high in transgenic lines (CD lines), roughly correlated with the expression levels of the transgene.

### EXAMPLE 2

Tomato CDI-Expressing Transgenic Potato Lines Exhibit Normal Growth and Development Rates

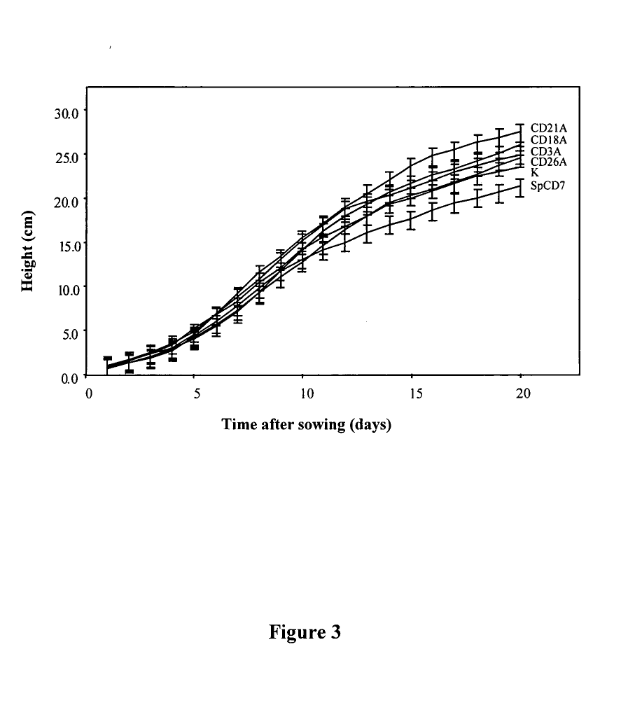

Growth parameters of selected CDI-expressing plants [chosen based on their content in tomato CDI; see. FIG. 2] were monitored daily for 20 days after tuber sowing, to detect eventual pleiotropic effects of recombinant CDI expressed in transgenic lines. Several individuals of each line were grown under greenhouse conditions under a 12 h/12 h L:D photoperiod and a light intensity of 200 μmol m⁻²s⁻¹, and monitored for various growth indicators. As shown on FIG. 3, growth rate, measured by plant's height over time, was not influenced by the inlibitor for all lines tested, even those expressing high amounts of cdi transcripts. Similarly, other parameters including tuber's germination time, number of leaves per plant, stem diameter and length of stem internodes were similar for all lines after 20 days (Table 1), indicating no visible pleiotropic effects of tomato CDI accumulated in the cytosol of transgenic potato cells, and no negative effect on the overall development of the transformed plants.

### EXAMPLE 3

Tomato CDI Accumulation in Transgenic Potato Leads to Increased Total Protein Content in Leaves

Considering that tomato CDI possesses protease inhibitory activity that may cause interference with endogenous proteases in potato, it was hypothesized that the expression of its cDNA-encoding sequence in the cytosol of transgenic potato plants may affect normal proteolysis of some endogenous proteins, resulting in an alteration of total protein composition and content. To assess this hypothesis, total protein content of fifteen potato clones selected based on expression of the cdi transgene (see Example I), and grown under greenhouse conditions, was determined in the 4^{th} leaf. Leaf proteins were extacted as described earlier (Cloutier et al. 2000), and protein contents were determined according to Bradford (1976), with bovine serum albumin as a standard.

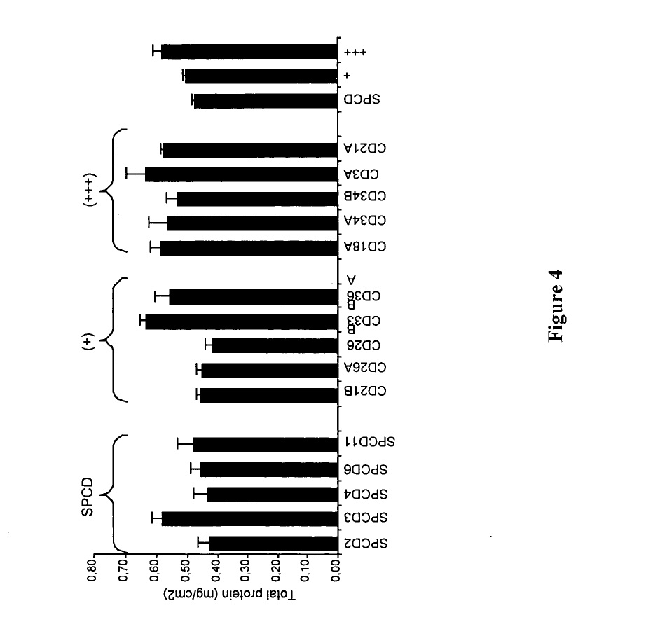

The results presented in FIG. 4A show that potato lines expressing CDI at a relatively high level (group “+++”) have about 20% more protein per leaf area than controls. For the growth conditions used, there were no significant differences in total protein content between group “+” and group SPCD (FIG. 4). Overall these results demonstrate that the expression of recombinant tomato CDI in the cytosol of potato leaf cells, while showing no significant effect on the plant's growth and development, caused a metabolic interference in planta presumably by interfering with endogenous proteinases, resulting in an increased protein concentration.

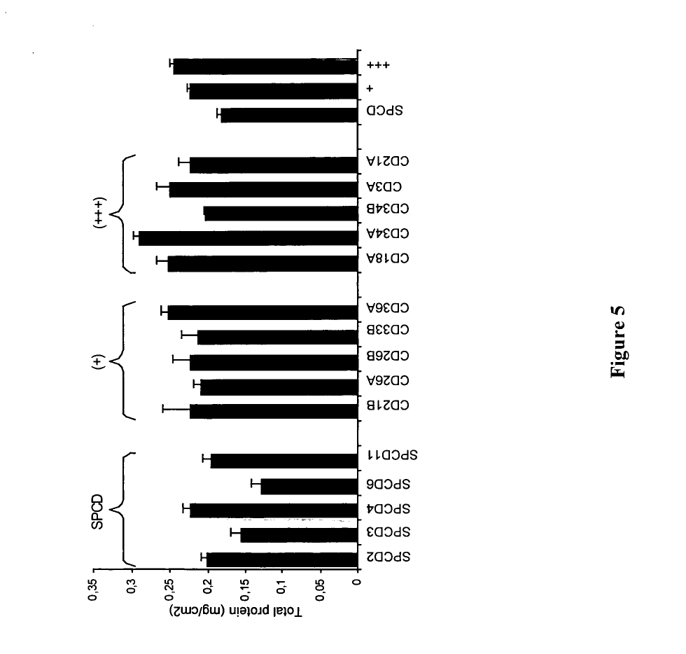

When potato plants were grown under low light intensities, leaf protein content decreased in the fifteen clones analysed, but leaf protein content in group “+++” was by about 35% higher than in group “SPCD” (FIG. 5). It is known from the art that plant growth conditions limiting carbon fixation may result in a reallocation of nitrogen in plants, triggered by the proteolysis of pre-existing endogenous proteins. Recombinant tomato CDI expressed in potato could modulate this process, thereby leading to increased protein content.

Finally, while the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

TABLE 1
Morphological parameters of control and CDI-expressing potato
clones^{a}

| Clone | Internode | Germination | Number of | Stem |
|---|---|---|---|---|
| (cm)^{b} | length (cm)^{c} | time (days) | leaves | diameter |
| Control (K) | 1.1 ± 0.2 | 13.0 ± 6.0 | 10.4 ± 1.3 | 0.75 ± 0.07 |
| SpCD7 | 1.1 ± 0.2 | 14.2 ± 5.5 | 10.8 ± 3.3 | 0.80 ± 0.11 |
| CD26A | 1.1 ± 0.3 | 9.6 ± 3.0 | 8.9 ± 1.6 | 0.76 ± 0.13 |
| CD18A | 1.4 ± 0.3 | 9.1 ± 3.7 | 10.0 ± 1.6 | 0.78 ± 0.12 |
| CD21A | 0.7 ± 0.2 | 9.4 ± 1.2 | 12.1 ± 3.5 | 0.56 ± 0.11 |
| CD3A | 1.1 ± 0.3 | 11.5 ± 3.0 | 10.2 ± 1.2 | 0.83 ± 0.07 |

^{a}Data obtained 20 days after tuber sowing. Each datum is the mean of 6 values ± se. No significant differences between clones for each parameter tested (ANOVA; P < 0.05).
^{b}Measured at the 3^{rd}-leaf internode.
^{c}Estimated as the distance between the 3^{rd} and 4^{th} nodes, from the apex.

### REFERENCES

Barry & Kishmore. U.S. Pat. No. 5,463,175
Bradford M M. 1976. A rapid and sensitive method for the quantitation of microgram quantities of protein utilizing the principle of protein-dye binding. Anal. Biochem. 72: 248-254.
Brunelle F, Cloutier C, Michaud D (2004) Colorado potato beetles compensate for tomato cathepsin D inhibitor expressed ill transgenic potato. Arch. Insect Biochem. Physiol. (in press).
Brunelle F, Nguyen-Quoc B, Cloutier C, Michaud D. 1999. Protein hydrolysis by Colorado potato beetle (Leptinotarsa decemlineata Say: Chrysomelidae) digestive proteases: the catalytic role of cathepsin D. Arch Insect Biocleem Physiol 42: 88-98.
Callis J (1995) Regulation of protein degradation. Plant Cell 7: 845-857.
Chistyakova L G, Antonov V K (1990) Regulation of intracellular proteolysis in Escherichia coli cells by antisens mRNAs of the lon gene. Biomed. Sci. 1: 359-365.
Cloutier C, Jean C, Fournier M, Yelle S, Michaud D. 2000. Adult Colorado potato beetles, Leptinotarsa decemlineata compensate for nutritional stress on oryzacystatin I-transgenic potato plants by hypertrophic behavior and over-production of insensitive proteases. Arch Insect Biochem Physiol 44: 69-81.
Cregg J M, Vedvick T S, Raschke W C (1993) Recent advances in the expression of foreign genes in Pichia pastoris. Biotechnology 11: 905-910.
Daniell H, Streatfield S (2001) Medical molecular farming: production of antibodies, biopharmaceuticals and edible vaccines in plants. Trends Plant Sci. 6: 219-226.
Doran P M (2000) Foreign protein production in plant tissue cultures. Curr. Opin. Biotechnol. 11: 199-204.
Edwards K, Johnstone C, Thompson C. 1991. A simple and rapid method for the preparation of plant genomic DNA for PCR analysis. Nucleic Acids Res. 19:1349.
Emr S D (1990) Heterologous gene expression in yeast. Meth. Enzymol. 185: 231-233.
Estelle M (2001) Proteases and cellular regulation in plants. Cwr. Opin. Plant Biol. 4: 254-260.
Fischer A, Emans N (2000) Molecular farming of pharmaceutical proteins. Transgenic Res. 9: 279-299.
Glotzner M, Murray A, Kirschner M W (1991) Cyclin is degraded by the ubiquitin pathway. Nature 349: 132-138.
Gottesman S (1990) Minimizing proteolysis in Escherichia coli: Genetic solutons. Methods Enzymol. 185: 119-129.
Hood B E, Woodard S L, Horn M E (2002) Monoclonal antibody manufacturing in transgenic plants—myths and realities. Curr. Opin. Biotechnol. 13: 630-635.
Jones B A, Hatfiled R D, Muck R E (1995) Crop quality and utilization. Characterization of proteolysis in alfalfa and red clover. Crop Sci. 35: 537-541.
Kohler G O, Wildman S G, Jorgensen N A, Enocian R V, Bray W J (1978) Leaf protein in relation to forage crop production and utilization. In Milner M, Scrimshaw N S, Wand D T, eds. Protein resources and technology, AVI Publishing Co, Inc, West Port, CT, 1978.
Kusnadi A R, Nikolov Z L, Howard J A (1997) Production of recombinant proteins in transgenic plants: practical considerations. Biotechnol. Bioeng. 56: 473-484.
Logemann I, Schell J. Willmitzer L. 1987. Improved method for the isolation of RNA from plant tissues. Anal. Biochem. 163:16-20.
Ma J K, Drake P M, Christou P (2003) The production of recombinant pharmaceutical proteins in plants. Nat. Rev. Genet. 4: 794-805.
McDonald P (1981) The biochemistry of silage. John Wiley & Sons, Ltd., New York.
McElroy et al. 1990, U.S. Pat. No. 5,463,175
Meerman H J, Georgiou G (1994) Construction and characterization od a set of E. coli strains deficient in all known loci affecting the proteolytic stability of secreted recombinant proteins. Biotechnology 12: 1107-1110.
Michaud D (2000) Recombinant protease iniibitors in plants. Landes Bioscience/Eurekah.com, Georgetown TX, 241 pp.
Michaud D & S Yelle. 2000. Production of useful protease inhibitors in plants. In Michaud D, ed: Recombinant Protease Inhibitors in Plants, Biotechnology Intelligence Unit Series, Landes Bioscience/Eurekah.com, Georgetown TX, pp. 191-202.
Michaud D, Vrain T C, Gomord V & L Faye. 1998. Stability of recombinant proteins in plants. In Cunningham C & A Porter, eds: Methods in Biotechnology, Vol. 3: Recombinant Proteins from Plants—Production of Clinically Useful Compounds. Humana Press, Totowa N.J., pp. 177-188.
Simon M-A, Nguyen-Quoc B, Michaud D (2000) Metabolic interference in potato tubers genetically-transformed with a cystatin-encoding cDNA sequence. Sixth International Congress of the International Society for Plant Molecular Biology, Quebec Q C, June, 18-24.
Rawn J D (1989) Biosynthesis of amino acids and related biosynthetic processes. In: Biochemistry. Neil Patterson Publ., Burlington NC, pp. 581-625.
Van der Vyver C, Schneideret J, Driscoll S, Turner J, Kunert K, Foyer C H (2003) Oryzacystatin I expression in transformed tobacco produces a conditional growth phenotype and enhances chilling tolerance. Plant Biotechnol. J. 1: 101-112.
Varshavsky A (1987) The N-end rule. Cell 69: 725-735.
Viersfra R D (1996) Protcolysis in plants: mechanism and fucntions. Plant Mol. Biol. 32: 275-302.
Wandelt C I, Khan M R I, Craig S et al. (1992) Vicilin with carboxy-terminal KDEL is retained in the endoplasmic-reticulum and accumulates to high-levels in the leaves of transgenic plants. Plant J. 2: 181-192.
Weber H, Rolletschek H, Heim U, Golombeck S, Gubatz S, Wobus U (2000) Antisens-inhibition of ADP-glucose pyrophosphorylase in developing seeds of Vicia narbonensis moderately decreases starch but increase protein content and affects seed maturation. Plant J. 24: 3343.
Wenzler H, Mignery G, May G, Park W (1989) A rapid and efficient transformation method for the production of large numbers of transgenic potato plants. Plant Sci. 63:79-85.
Werner R, Cuitton M-C, Mühlbach H-P (1993) Nucleotide sequence of a cathepsin D inhibitor protein from tomato. Plant Physiol. 103:1473.

1. A method for increasing protein content in a plant cell, said method comprising inducing said plant cell to recombinantly produce a modifier of protein metabolism.

2. The method of claim 1, wherein said modifier alters the overall synthesis/degradation balance of proteins in said plant cells.

3. The method of claim 2 wherein said alteration of protein metabolism takes place in the cytoplasm of said cell.

4. The method of claim 1 wherein said step of inducing comprises:
transforming said cell with a genetic construct comprising a promoter operatively linked to a gene coding for said modifier of protein metabolism; and
submitting said plant cell to conditions compatible with active protein metabolism.

5. The method of claim 4, wherein said plant cell is episodically or genomically modified.

6. The method as claimed in claim 5, wherein said conditions comprise low light intensity.

7. The method of claim 2, wherein said modifier is a protease inhibitor.

8. The method of claim 7, wherein said protease inhibitor is selected from the group consisting of aspartate protease inhibitors, cysteine protease inhibitors, metalloprotease inhibitors, and serine protease inhibitors.

9. The method of claim 8, wherein said protease inhibitor is a cathepsin D inhibitor.

10. A plant cell or a plant in which is recombinantly produced a modifier of protein metabolism, thereby increasing protein content of said plant cell or plant.

11. The plant cell or plant as claimed in claim 13 wherein said modifier alters the overall synthesis/degradation balance of proteins in said plant cells.

12. A transgenic plant transformed with a genetic construct comprising a promoter operatively linked to a gene encoding a modifier of protein metabolism, and wherein said transgenic plant produces higher levels of protein relative to the non-transformed plant.

13. The transgenic plant of claim 12 wherein said modifier is a protease inhibitor.

14. The transgenic plant of claim 13, wherein said protease inhibitor is selected from the group consisting of aspartate protease inhibitors, cysteine protease inhibitors, metalloprotease inhibitors, and serine protease inhibitors.

15. The transgenic plant of claim 14, wherein said protease inhibitor is a cathepsin D inhibitor.

16. The method of claim 1, wherein said protein content is increased from about 10 to about 50% when compared to the protein content of a plant cell in which said modifier is not recombinantly produced.


SEQUENCE LISTING (4 sequences)

SEQ ID NO: 1 - DNA, 30 bp, Artificial Sequence
  primer_bind (1)...(30): Sense nptii primer
actgaagcgggaagggactggctgctattg

SEQ ID NO: 2 - DNA, 29 bp, Artificial Sequence
  primer_bind (1)...(29): Antisense nptii primer
gataccgtaaagcacgaggaagcggtcag

SEQ ID NO: 3 - DNA, 50 bp, Artificial Sequence
  primer_bind (1)...(50): Sense cdi primer
aaggatccgtgcacaaaagatggctgcttctcctaaacctaatccagtac

SEQ ID NO: 4 - DNA, 40 bp, Artificial Sequence
  primer_bind (1)...(40): Antisense cdi primer
aacccgggaagccgagactttcttgaagtagacccccaag